(19) United States
(12) Patent Application Publication
(10) Pub. No.: US 2025/0360138 A1
(43) Pub. Date: Nov. 27, 2025

(54) MAVELERTINIB AS A TREATMENT FOR GIARDIASIS

(71) Applicants: University of Washington, Seattle, WA (US); The Scripps Research Institute, La Jolla, CA (US)

(72) Inventors: Kayode K. Ojo, Seattle, WA (US); Erkang Fan, Seattle, WA (US); Samantha A. Michaels, Seattle, WA (US); Melissa Love, La Jolla, CA (US); Case McNamara, La Jolla, CA (US)

(73) Assignees: University of Washington, Seattle, WA (US); The Scripps Research Institute, La Jolla, CA (US)

(21) Appl. No.: 18/872,524

(22) PCT Filed: Jun. 5, 2023
(86) PCT No.: PCT/US2023/067941

§ 371 Date: Dec. 6, 2024

Related U.S. Application Data
(60) Provisional application No. 63/349,446, filed on Jun. 6, 2022.

(51) Int. Cl. A61K 31/52; A61P 33/02

(52) U.S. Cl. CPC A61K 31/52; A61P 33/02

(57) ABSTRACT

Use of the epidermal growth factor receptor tyrosine kinase inhibitor (EGFR-TKI), mavelertinib, as a treatment for giardiasis in humans, veterinary animals, and livestock is described. Mavelertinib demonstrates a high selectivity index for Giardia compared to mammalian cells and therefore provides an effective and safe treatment for giardiasis. Therapeutically effective amounts for oral administration are also described.

### CROSS-REFERENCE TO RELATED APPLICATIONS

This application is a U.S. National Phase Patent Application based on International Patent Application No. PCT/US2023/067941, filed on Jun. 5, 2023, which claims priority to U.S. Provisional Patent Application No. 63/349,446 filed Jun. 6, 2022, both of which are incorporated herein by reference in their entirety as if fully set forth herein.

### STATEMENT REGARDING FEDERALLY SPONSORED RESEARCH OR DEVELOPMENT

This invention was made with government support under Grant No. R21 A1140881, awarded by the National Institutes of Health. The government has certain rights in the invention.

### FIELD OF THE DISCLOSURE

The current disclosure provides mavelertinib as a treatment for giardiasis in humans, veterinary animals, and livestock.

### BACKGROUND OF THE DISCLOSURE

Giardiasis, a parasitic infection of the small intestines of humans or animals, is caused by the parasite Giardia duodenalis (also known as Giardia lamblia or Giardia intestinalis). This prevalent parasitic pathogen has a global impact, affecting over one million people per year. The common transmission of giardiasis is through the consumption of food or drinking water contaminated with Giardia duodenalis.

There are two forms of Giardia duodenalis: a trophozoite (the active form) and a cyst (the inactive form). Ten or more ingested Giardia cyst parasites lead to infection. Within the pH and enzymatic environment of the small intestine, the inactive Giardia cyst develops into the active form of the parasite trophozoite. The trophozoite attaches to the intestinal wall via a suction force. Once the trophozoite is attached to the intestinal wall, infection occurs. Subsequently, a human or animal begins to experience giardiasis symptoms, including diarrhea, gas, stomach pain, nausea, vomiting, and/or dehydration. Trophozoite (which cannot live outside of the body) then begin to produce Giardia cysts, which are expelled from the body in the feces, leading to the potential spread of infection. Giardia cysts can live for prolonged periods outside of the body.

There are several treatments for Giardia infection that are available, however, these treatments are associated with drawbacks such as adverse side effects or incomplete efficacy, as explained in more detail below.

### SUMMARY OF THE DISCLOSURE

The current disclosure provides use of mavelertinib as a treatment for giardiasis in humans, veterinary animals, and livestock.

### BRIEF DESCRIPTION OF THE DRAWINGS

Some of the drawings submitted herein may be better understood in color. Applicants consider the color versions of the drawings as part of the original submission and reserve the right to present color images of the drawings in later proceedings.

FIG. 1. Flowchart of screening workflow and selection of high-value hits for resupply. The primary screening against G. lamblia GS-clone H7-strain (Assemblage B) was at a final concentration of 5 μM. The majority of the compounds that reconfirmed in dose response were either cytotoxic, had a chemical scaffold similar to the standard of care (SOC), or were unavailable for resupply, leaving only 2 selective compounds with a novel chemotype available for resupply. Based on reconfirmation in the G. lamblia CBG99 luciferase strain, Mavelertinib and pelletierine were selected for resupply for testing in the mouse model of giardiasis.

FIG. 2. Chemical structure of selective hits with potential novel mechanism of action/chemotypes. The 6 primary hits, PR-104, CHF-6001, Mavelertinib, propyl red, caroverine and pelletierine reconfirmed in follow-up assay have Anti-Giardia EC₅₀ of 1.98 μM, 2.03 μM, 2.34 μM, 5.78 μM, 13.70 μM and 20.30 μM respectively. Selectivity index (SI) (defined as CC₅₀/EC₅₀ against G. lamblia GS-clone 7 trophozoites versus toxicity in mammalian cell cultures) was >13, >12, >11, >4, >2, and >1 for PR-104, CHF-6001, Mavelertinib, propyl red, caroverine and pelletierine, respectively. The chemical structure of Mavelertinib shows the reactive, covalently-binding acrylamide chemical functional group.

FIGS. 3A-3C. 3A) Noninvasive imaging of G. lamblia CBG99 strain trophozoite growth in mice. Treatment started 6 days post-infection after confirmation of infection. Mice were treated with Mavelertinib (Mav) at 50 mg/kg×1 (one day), 20 mg/kg orally once per day (QD)×2 (two days), 5 mg/kg QD×2 and 2.5 mg/kg QD×2. Metronidazole (Met), dosed at 20 mg/kg QD×3 (three days), was used as a control. Positive controls included Compound-1717 at 50 mg/kg QD×2 (two days), while a group of mice dosed with the blank vehicle (VEH) served as the untreated control. (3B) Radiance plot and mouse images show absence of luminescence signal after a single 50 mg/kg treatment, 20 mg/kg QD×2, and 5 mg/kg QD×2 of Mavelertinib as well as 50 mg/kg QD×2 of 1717 relative to the untreated or metronidazole treated controls. (3C) A plot of the average measured photon (radiance) of bioluminescence G. lamblia CBG99 trophozoites in 1 mg/kg Mavelertinib treated and untreated mice. In this experiment, in vivo efficacy of Mavelertinib (plot with circles) dosed at 1 mg/kg QD on days 6-9 p.i. against G. lamblia CBG99 infected IFN-γ-knockout (KO) mice was measured. Treatment control for the study included 20 mg/kg metronidazole (plot with squares), vehicle treated infected mice and uninfected background controls (BKGD). Mavelertinib dosed at 1 mg/kg did not clear the infection after 4 doses.

FIG. 4. Pharmacokinetic analysis of blood at treatment concentrations. The time at which maximum plasma concentration (Cₘₐₓ) is observed (Tₘₐₓ) was 0.5 h in all cases. The 50 mg/kg dose had an average Cₘₐₓ of 9.3 μM at 0.5 hours post dose. The 20 mg/kg and 5 mg/kg Cₘₐₓ were 3.5 μM and 0.63 μM, respectively. For the 1 mg/kg QD treatment group, Mavelertinib was barely detectable in the plasma samples at all time points sampled. All dosage groups had plasma concentrations below the detection limit at 24 h post dose.

FIG. 5. Radiance plot and mouse images before and after treatment with compound-1717: Compound-1717 was dosed at 50 mg/kg×2 days, 20 mg/kg QD×2 days, 5 mg/kg QD×3 days, 2.5 mg/kg QD×3 days. The infection was cleared in the 50 and 20 mg/kg QD×2 days groups, while the 5 mg/kg and 2.5 mg/kg QD×3 days did not clear the infection.

FIG. 6. Selective, reconfirmed hits that were available for resupply. Only two compounds were resupplied for follow-up testing based on the chemical scaffolds being distinct from standard of care.

FIG. 7. Results of epidermal growth factor receptor tyrosine kinase inhibitors (EGFR-TKIs) cell growth inhibition assay against G. lamblia strains (EC₅₀), cytotoxicity against CRL-8155 and HepG2 (CC₅₀) and selectivity index. *The ratio of efficacy of each inhibitor against G. lamblia GS-clone 7 trophozoites versus toxicity in mammalian cell cultures was used to calculate the selectivity index (SI), where SI is defined as CC₅₀/EC₅₀. ND stands for not done.

### DETAILED DESCRIPTION

Giardia lamblia, a waterborne intestinal parasite, is a major contributor to the global burden of diarrheal diseases. Its oral-fecal mode of transmission could be zoonotic or, more commonly, human-to-human. The natural anatomic niche of Giardia trophozoites in humans is the small intestine, particularly the duodenum. Giardia replicates in the small intestine and attaches to intestinal cells through the ventral disk, resulting in the impairment of the host's ability to absorb necessary nutrients effectively. There are conflicting conclusions from studies of the impact of G. lamblia on children in developing countries. Some suggest that it is particularly profound since the disease can contribute to malnutrition, growth retardation, and poor cognitive function, while others claim that Giardia infection is protective against moderate to severe diarrhea. Similarly, the long-term consequences of giardiasis are only now starting to be understood. Half of infections can be asymptomatic, yet there is increasing evidence that chronic infections can lead to colitis, food allergies, long term Irritable Bowel Syndrome, and chronic fatigue. Despite the enormous public health concern posed by the high prevalence of giardiasis among children in economically challenged regions, treatment of symptomatic and asymptomatic giardiasis is often constrained by limited therapeutic options and/or development of resistance to available treatments. Approved therapies for giardiasis include the broad-spectrum antimicrobials metronidazole and tinidazole. However, a significant proportion of clinical cases involve metronidazole—(and presumably tinidazole—) resistant Giardia.

In addition to the widely discussed limitations like reduced efficacy and adverse side-effects, many available giardiasis drugs also have broad spectrum antimicrobial activity, which may lead to the development of gut dysbiosis. Gut dysbiosis has the potential to be a severe complication of giardiasis treatment in malnourished children, a major population group for Giardia therapeutics. The urgency of the public health demand necessitates a concentrated focus on accelerated development of alternatives for clinical use against all Giardia strains, including ones with resistance to current treatments. Given its high prevalence in resource-limited regions, giardiasis is a neglected infectious disease that could benefit from an accelerated therapeutic development program based on drug repurposing platforms.

The current disclosure provides use of mavelertinib as a treatment for giardiasis in humans, veterinary animals, and livestock. Mavelertinib is a mutant epidermal growth factor receptor tyrosine kinase inhibitor (EGFR-TKI) in development as a treatment for non-small cell lung cancer. Mavelertinib (1-[(3R,4R)-3-[({5-chloro-2-[(1-methyl-1H-pyrazol-4-yl)amino]-7H-pyrrolo[2,3-d]pyrimidin-4-yl}[1,2]oxy)methyl]-4-methoxypyrrolidin-1-yl]propan-1-one) has the structure:

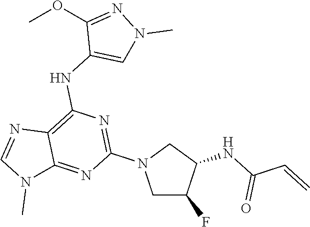 [SMILES: C=CC(=O)N[C@H]1CN(c2nc(Nc3cn(C)nc3OC)c3ncn(C)c3n2)C[C@@H]1F]

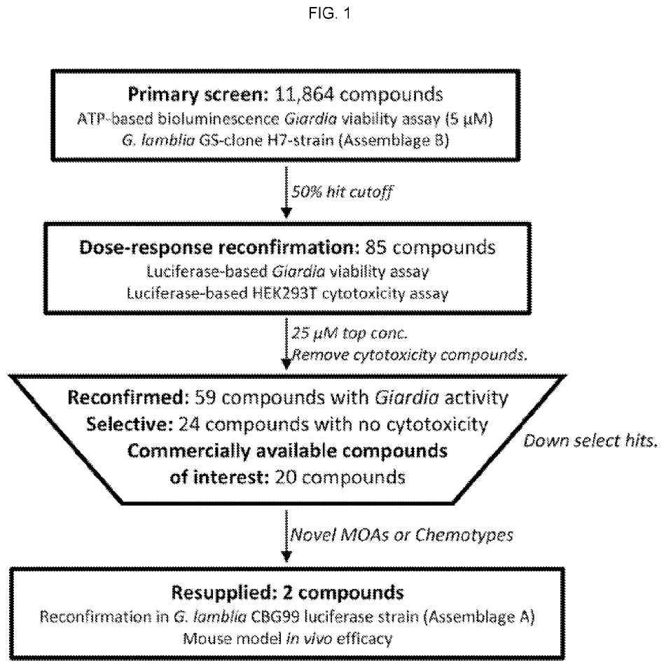

Mavelertinib was selected as a hit from the 11,865 ReFRAME compound library screen against G. lamblia (FIG. 1). It underwent mouse pharmacokinetic analysis in female BALB/c mice with no sign of toxicity and was subsequently assayed in in vivo efficacy studies using the G. lamblia CBG99 luciferase strain as described below. Using the G. lamblia CBG99 strain has established techniques for longitudinal, noninvasive, quantitative measurement of treatment effects on total parasite count. The method relies on a stable constitutive luciferase reporter, CBG99 (Click beetle green), that correlates with parasite load in vitro and in vivo. Successful infection in mice is clearly detectable by 5 days post infection (p.i.) using IVIS imaging and persists for more than 2 weeks. Only mice with confirmed infections are used in experiments. Untreated control mice will have persistent infection, whereas all adequately treated mice are cleared of infection.

Aspects of the current disclosure are now described with additional detail and options as follows: (i) Compositions for Administration; (ii) Methods of Use; (iii) Exemplary Embodiments; (iv) Experimental Example; and (v) Closing Paragraphs. These headings are provided for organizational purposes only and do not limit the scope or interpretation of the disclosure.

### (i) Compositions for Administration

Mavelertinib can be formulated for administration to subjects in one or more pharmaceutically acceptable carriers. Pharmaceutically acceptable salts, stereoisomers, tautomers, atropisomers, hemisalts, and/or solvates of mavelertinib can also be used.

Pharmaceutically acceptable salts of mavelertinib include the acid addition and base addition salts thereof.

Suitable acid addition salts are formed from acids which form non-toxic salts. Examples of suitable acid addition salts, i.e., salts containing pharmacologically acceptable anions include the acetate, acid citrate, adipate, aspartate, benzoate, besylate, bicarbonate/carbonate, bisulphate/sulphate, bitartrate, borate, camsylate, citrate, cyclamate, edisylate, esylate, ethanesulfonate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methanesulfonate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, p-toluenesulfonate, tosylate, trifluoroacetate and xinofoate salts.

Additional embodiments relate to base addition salts of mavelertinib. Suitable base addition salts are formed from bases which form non-toxic salts. Examples of suitable base salts include the aluminum, arginine, benzathine, calcium, choline, diethylamine, diolamine, glycine, lysine, magnesium, meglumine, olamine, potassium, sodium, tromethamine and zinc salts.

Mavelertinib is basic in nature and capable of forming a wide variety of salts with various inorganic and organic acids. The acids that may be used to prepare pharmaceutically acceptable acid addition salts of such basic compounds described herein are those that form non-toxic acid addition salts, e.g., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

The chemical bases that may be used as reagents to prepare pharmaceutically acceptable base salts of mavelertinib that are acidic in nature are those that form non-toxic base salts with such compounds. Such non-toxic base salts include those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Reference to mavelertinib includes all stereoisomers (e.g., cis and trans isomers) and all optical isomers (e.g., R and S enantiomers), as well as racemic, diastereomeric and other mixtures of such isomers. While all stereoisomers are encompassed within the scope of the disclosure, one skilled in the art will recognize that particular stereoisomers may be preferred.

In some embodiments, mavelertinib can exist in several tautomeric forms, including the enol and imine form, and the keto and enamine form and geometric isomers and mixtures thereof.

All such tautomeric forms are included within the scope of the present embodiments. Tautomers exist as mixtures of a tautomeric set in solution. In solid form, usually one tautomer predominates.

The present embodiments also include atropisomers of mavelertinib. Atropisomers refer to compounds that can be separated into rotationally restricted isomers.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

The term “solvate” is used herein to describe a molecular complex including mavelertinib and one or more pharmaceutically acceptable solvent molecules, for example, ethanol.

Mavelertinib may also exist in unsolvated and solvated forms. Accordingly, some embodiments relate to the hydrates and solvates of mavelertinib.

Included within the scope of the present embodiments are all stereoisomers, geometric isomers and tautomeric forms of mavelertinib, including compounds exhibiting more than one type of isomerism, and mixtures of one or more thereof. Also included are acid addition or base salts wherein the counterion is optically active, for example, d-lactate or I-lysine, or racemic, for example, dI-tartrate or dI-arginine.

Cis/trans isomers may be separated by techniques well known to those skilled in the art, for example, chromatography and fractional crystallization.

Techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Because giardiasis is an infection of the gastrointestinal (GI) tract, particular embodiments include oral compositions.

Exemplary excipient classes for oral compositions include binders, buffers, chelators, coating agents, colorants, complexation agents, diluents (i.e., fillers), disintegrants, emulsifiers, flavoring agents, glidants, lubricants, preservatives, releasing agents, surfactants, stabilizing agents, solubilizing agents, sweeteners, thickening agents, wetting agents, and vehicles.

Binders are substances used to cause adhesion of powder particles in granulations.

Exemplary binders include acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, and polyethylene glycol.

Colorants may be included in the oral compositions to impart color to the composition.

Exemplary colorants include grape skin extract, beet red powder, beta carotene, annato, carmine, turmeric, and paprika. Additional colorants include FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide.

Diluents, also termed “fillers”, can enhance the granulation of oral compositions and/or be used to increase the bulk of a tablet so that a practical size is provided for compression.

Exemplary diluents include cellulose, microcrystalline cellulose, sucrose, powdered sugar, talc, sodium chloride, sodium dioxide, titanium oxide, dicalcium phosphate, dihydrate, calcium sulfate, calcium carbonate, alumina, kaolin, starches, lactose and polyols of less than 13 carbon atoms, such as mannitol, xylitol, sorbitol, maltitol and pharmaceutically acceptable amino acids, such as glycin.

Disintegrants also may be included in the oral compositions in order to facilitate dissolution. Disintegrants, including permeabilizing and wicking agents, are capable of drawing water or saliva up into the oral compositions which promotes dissolution from the inside as well as the outside of the oral compositions. Such disintegrants, permeabilizing and/or wicking agents that may be used include starches, such as corn starch, potato starch, pre-gelatinized and modified starches thereof, cellulosic agents, such as Ac-di-sol, montmorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose.

Dissolution of the oral compositions may be facilitated by including relatively small particles sizes of the ingredients used.

Exemplary dispersing or suspending agents include acacia, alginate, dextran, fragacanth, gelatin, hydrogenated edible fats, methylcellulose, polyvinylpyrrolidone, sodium carboxymethyl cellulose, sorbitol syrup, and synthetic natural gums.

Exemplary emulsifiers include acacia, lecithin, carrageenan, guar gum, xantham gum, polysorbates, cellulose (e.g., carboxymethylcellulose), monoglycerides of fatty acids, diglycerides of fatty acids, sucrose esters, sucroglycerides, polyglycerol esters of fatty acids, polyglycerol polyricinoleate, stearoyl lactylates, and sorbitan esters.

Flavorants are natural or artificial compounds used to impart a pleasant flavor and often odor to oral compositions. Exemplary flavorants include, natural and synthetic flavor oils, flavoring aromatics, extracts from plants, leaves, flowers, and fruits and combinations thereof. Such flavorants include anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wild berry, date, blueberry, kiwi, strawberry, raspberry, cherry, plum, pineapple, and apricot. In particular embodiments, flavorants that may be used include natural berry extracts and natural mixed berry flavor, as well as citric and malic acid.

Glidants improve the flow of powder blends during manufacturing and minimize oral composition weight variation. Exemplary glidants include silicon dioxide, colloidal or fumed silica, magnesium stearate, calcium stearate, stearic acid, cornstarch, and talc.

Lubricants are substances used in oral compositions that reduce friction during composition compression. Exemplary lubricants include stearic acid, calcium stearate, magnesium stearate, zinc stearate, stearic acid, talc, mineral and vegetable oils, benzoic acid, poly(ethylene glycol), glyceryl behenate, stearyl fumarate, and sodium lauryl sulfate.

Exemplary preservatives include methyl p-hydroxybenzoates, propyl p-hydroxybenzoates, and sorbic acid. Exemplary preservative can also include benzalkonium chloride, benzethonium chloride, or chlorobutanol.

Exemplary sweeteners include aspartame, dextrose, fructose, high fructose corn syrup, maltodextrin, monoammonium glycyrrhizinate, neohesperidin dihydrochalcone, potassium acesulfame, saccharin sodium, stevia, sucralose, and sucrose. Sweeteners also include white sugar, corn syrup, sorbitol (solution), maltitol (syrup), oligosaccharide, isomaltooligosaccharide, sucrose, fructose, lactose, glucose, lycasin, xylitol, D-mannose, lactitol, erythritol, mannitol, isomaltose, dextrose, polydextrose, dextrin, compressible cellulose, compressible honey, compressible molasses and mixtures thereof.

Particular embodiments include swallowable compositions. Swallowable compositions are those that do not readily dissolve when placed in the mouth and may be swallowed whole without chewing or discomfort. U.S. Pat. Nos. 5,215,754 and 4,374,082 describe methods for preparing swallowable compositions. In particular embodiments, swallowable compositions may have a shape containing no sharp edges and a smooth, uniform and substantially bubble free outer coating.

To prepare swallowable compositions, each of the ingredients may be combined in intimate admixture with a suitable carrier according to compounding techniques. In particular embodiments of the swallowable compositions, the surface of the compositions may be coated with a polymeric film. Such a film coating has several beneficial effects. First, it reduces the adhesion of the compositions to the inner surface of the mouth, thereby increasing the subject's ability to swallow the compositions. Second, the film may aid in masking the unpleasant taste of certain ingredients. Third, the film coating may protect the compositions from atmospheric degradation. Polymeric films that may be used in preparing the swallowable compositions include vinyl polymers such as polyvinylpyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac, and acacia.

In particular embodiments, the oral compositions may include chewable compositions. Chewable compositions are those that have a palatable taste and mouthfeel, are relatively soft and quickly break into smaller pieces and begin to dissolve after chewing such that they are swallowed substantially as a solution.

U.S. Pat. No. 6,495,177 describes methods to prepare chewable compositions with improved mouthfeel. U.S. Pat. No. 5,965,162, describes kits and methods for preparing comestible units which disintegrate quickly in the mouth, especially when chewed.

In order to create chewable compositions, certain ingredients should be included to achieve the attributes just described. For example, chewable compositions should include ingredients that create pleasant flavor and mouthfeel and promote relative softness and dissolvability in the mouth. Sweeteners as described earlier can be used to achieve mouthfeel and palatability characteristics. Furthermore, fondant or gums and fatty materials can be used to promote mouthfeel, relative softness, and dissolvability.

Fondant or gums such as gelatin, agar, arabic gum, guar gum, and carrageenan may be added to improve the chewiness of the compositions. Fatty materials that may be used include vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used to enhance the texture, the mouthfeel, or both of chewable compositions. By “enhance the texture” it is meant that the alkyl polysiloxane improves one or more of the stiffness, the brittleness, and the chewiness of the chewable composition, relative to the same preparation lacking the alkyl polysiloxane. By “enhance the mouthfeel” it is meant that the alkyl polysiloxane reduces the gritty texture of the chewable composition once it has liquefied in the mouth, relative to the same preparation lacking the alkyl polysiloxane.

Alkyl polysiloxanes generally include a silicon and oxygen-containing polymeric backbone with one or more alkyl groups pending from the silicon atoms of the backbone. Depending upon their grade, they can further include silica gel. Alkyl polysiloxanes are generally viscous oils.

Exemplary alkyl polysiloxanes that can be used in swallowable, chewable or dissolvable compositions include monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP.

Simethicone USP contains not less than 90.5% by weight (CH₃)₃—Si{OSi(CH₃)₂}CH₃ in admixture with 4.0% to 7.0% by weight SiO₂.

To prevent the stickiness that can appear in some chewable compositions and to facilitate conversion of the active ingredients to emulsion or suspension upon taking, the compositions may further include emulsifiers such as glycerin fatty acid ester, sorbitan monostearate, sucrose fatty acid ester, lecithin and mixtures thereof. In particular embodiments, one or more of such emulsifiers may be present in an amount of 0.01% to 5.0%, by weight of the administered compositions. If the level of emulsifier is lower or higher, in particular embodiments, an emulsification cannot be realized, or wax value will rise.

Liquid preparations for oral administration may take the form of, for example, solutions, syrups or suspensions, or they may be presented as a dry product for reconstitution with water or other suitable vehicles before use.

In addition to those described above, any appropriate fillers and excipients may be utilized in preparing the swallowable, chewable and/or dissolvable compositions or any other oral composition described herein so long as they are consistent with the described objectives.

Oral compositions also include edibles. Edibles refer to any product that can be consumed as a food or a drink. In some cases, edibles are made by infusion of mavelertinib into a foodstuff. Examples of edible foods appropriate for use include candy, a candy bar, bread, a brownie, cake, cheese, chocolate, cocoa, a cookie, gummy candy, a lollipop, a mint, a pastry, peanut butter, popcorn, a protein bar, rice cakes, yogurt, etc. While technically not edible, gums can also be used. Examples of edible drinks include beer, juice, flavored milk, flavored water, liquor, milk, punch, a shake, soda, tea, and water. In particular embodiments, edibles are made by combining mavelertinib with ingredients used to make an edible. Examples include butters and oils.

Exemplary oils include coconut oil, grape seed oil, olive oil, palm oil, papaya seed oil, peanut oil, sesame oil, sprouted wheat oil, wheat germ oil, or any combination thereof.

Oral compositions can be individually wrapped or packaged as multiple units in one or more packages, cans, vials, blister packs, or bottles of any size. Doses are sized to provide therapeutically effective amounts.

While oral dosage forms are preferred, the disclosure is not limited to these dosage forms.

For example, when formulated for injection, compositions can include saline, buffered saline, physiological saline, water, Hanks' solution, Ringer's solution, Normosol-R (Abbott Labs), glycerol, ethanol, sterile aqueous solutions, for example, aqueous propylene glycol, and combinations thereof. In particular embodiments, a carrier for infusion includes buffered saline with 5% HSA or dextrose. Additional isotonic agents include polyhydric sugar alcohols, including trihydric or higher sugar alcohols, such as glycerin, erythritol, arabitol, xylitol, sorbitol, or mannitol.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including mavelertinib. Various sustained-release materials have been established and are well known by those of ordinary skill in the art.

Sustained-release systems may, depending on their chemical nature, release mavelertinib following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

Carriers can additionally include pharmaceutically acceptable buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and compositions are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

Therapeutically effective amounts of mavelertinib within a composition can include at least 0.1% w/v or w/w mavelertinib; at least 1% w/v or w/w mavelertinib; at least 10% w/v or w/w mavelertinib; at least 20% w/v or w/w mavelertinib; at least 30% w/v or w/w mavelertinib; at least 40% w/v or w/w mavelertinib; at least 50% w/v or w/w mavelertinib; at least 60% w/v or w/w mavelertinib; at least 70% w/v or w/w mavelertinib; at least 80% w/v or w/w mavelertinib; at least 90% w/v or w/w mavelertinib; at least 95% w/v or w/w mavelertinib; or at least 99% w/v or w/w mavelertinib.

### (ii) Methods of Use

Methods disclosed herein include treating subjects (humans, veterinary animals (dogs, cats, reptiles, birds, etc.), livestock (horses, cattle, goats, pigs, chickens, etc.) and research animals (monkeys, rats, mice, fish, etc.)) with compositions disclosed herein. Treating subjects includes delivering therapeutically effective amounts. Therapeutically effective amounts include those that provide effective amounts, prophylactic treatments, and/or therapeutic treatments.

An “effective amount” is the amount of a composition necessary to result in a desired physiological change in the subject. For example, an effective amount can provide an anti-giardiasis effect. Effective amounts are often administered for research purposes. Effective amounts disclosed herein can cause a statistically-significant effect in an animal model or in vitro assay relevant to the assessment of giardiasis development or progression. In particular embodiments, effective amounts reduce giardiasis in a subject.

A “prophylactic treatment” includes a treatment administered to a subject who does not display signs or symptoms of giardiasis or displays only early signs or symptoms of giardiasis such that treatment is administered for the purpose of diminishing or decreasing the risk of developing giardiasis further. Thus, a prophylactic treatment functions as a preventative treatment against giardiasis or the worsening of giardiasis. In particular embodiments, prophylactic treatments reduce, delay, or prevent the development of giardiasis.

A “therapeutic treatment” includes a treatment administered to a subject who displays symptoms or signs of giardiasis and is administered to the subject for the purpose of diminishing or eliminating those signs or symptoms of giardiasis. The therapeutic treatment can reduce, control, or eliminate the presence or activity of giardiasis and/or reduce control or eliminate side effects of giardiasis.

Function as an effective amount, prophylactic treatment, or therapeutic treatment are not mutually exclusive, and in particular embodiments, administered dosages may accomplish more than one treatment type. Therapeutically effective amounts can be confirmed by observing a reduction in one or more symptoms of giardiasis such as diarrhea, gas, stomach pain, nausea, vomiting, cramps, flatulence, anorexia, malaise, fatigue, and/or dehydration

For administration, therapeutically effective amounts (also referred to herein as doses) can be initially estimated based on results from in vitro assays and/or animal model studies. Such information can be used to determine useful doses in subjects of interest more accurately. The actual dose amount administered to a particular subject can be determined by a physician, veterinarian, or researcher, taking into account parameters such as physical and physiological factors including target, body weight, stage of giardiasis, the severity of giardiasis, previous or concurrent therapeutic interventions, idiopathy of the subject and route of administration.

Useful doses can range from 0.1 to 150 mg/kg or from 2.5-5 mg/kg. Particularly useful doses can include 0.2 mg/kg, 0.4 mg/kg, 1.8 mg/kg, 2.6 mg/kg, 2.5 mg/kg, 5 mg/kg, 20 mg/kg, and 50 mg/kg. In humans, useful doses can range from 0.1 mg/kg to 2.6 mg/kg. Particularly useful doses in humans can include 0.2 mg/kg, 0.3 mg/kg, 0.4 mg/kg, 0.5 mg/kg, 0.6 mg/kg, 0.8 mg/kg, 1 mg/kg, 1.2 mg/kg, 1.4 mg/kg, 1.6 mg/kg, 1.9 mg/kg, or 2.6 mg/kg. Adverse side effects can emerge for doses over 150 mg (2.6-1.9 mg/kg). In particular embodiments, adverse side effects can emerge for doses over 150 mg per day for over 7 days. In particular embodiments, adverse side effects include diarrhea and skin toxicities.

The pharmaceutical composition may be in unit dosage forms suitable for single administration of precise dosages.

Therapeutically effective amounts can be achieved by administering single or multiple doses during the course of a treatment regimen. Particular embodiments include a high dose (e.g., 50 mg/kg) with fewer administrations (e.g., 1). Other embodiments can include a lower dose (e.g., 2.5 mg/kg) with more administrations (e.g., 1-5). Other dosing schedules may also be used given the goals of a therapy (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

Administration of the compositions can be affected by any method that enables delivery of the compounds to the site of action. These methods include oral routes, intraduodenal routes, and parenteral injection (including intravenous, subcutaneous, intramuscular, intravascular, or infusion).

As indicated, the compositions disclosed herein can be administered by, e.g., oral administration of a solid or liquid composition including mavelertinib, injection of a composition including mavelertinib, or local implantation of a sustained-release composition including mavelertinib.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (iii) Exemplary Embodiments

1. A method of treating giardiasis in a subject in need thereof including administering a therapeutically effective amount of a composition including mavelertinib to the subject, thereby treating giardiasis in the subject in need thereof.
2. The method of embodiment 1, wherein the administering is a single administration.
3. The method of embodiment 1, wherein the administering is two administrations.
4. The method of embodiment 3, wherein the two administrations are each on a consecutive day.
5. The method of embodiment 1, wherein the administering is three, four, five, six, or seven administrations.
6. The method of embodiment 5, wherein the three, four, five, six, or seven administrations are each on a consecutive day.
7. The method of any of embodiments 1-6, wherein the administering includes oral administration.
8. The method of any of embodiments 1-7, wherein the subject is a human, veterinary animal, or livestock.
9. The method of any of embodiments 1-8, wherein the human is a pediatric patient.
10. The method of embodiment 9, wherein the pediatric patient is less than 18 years of age.
11. The method of any of embodiments 1-8, wherein the human is an adult.
12. The method of embodiment 11, wherein an adult is 18 years of age or older.
13. The method of any of embodiments 9-12, wherein the therapeutically effective amount is 0.1 mg/kg-2.6 mg/kg.
14. The method of embodiment 13, wherein the therapeutically effective amount is 0.1 mg/kg-1.9 mg/kg.
15. The method of embodiment 13, wherein the therapeutically effective amount is 0.2 mg/kg-0.4 mg/kg.
16. The method of any of embodiments 13-15, wherein the therapeutically effective amount is 0.2 mg/kg, 0.3 mg/kg, 0.4 mg/kg, 0.5 mg/kg, 0.6 mg/kg, 0.8 mg/kg, 1 mg/kg, 1.2 mg/kg, 1.4 mg/kg, 1.6 mg/kg, 1.9 mg/kg, or 2.6 mg/kg.
17. The method of any of embodiments 1-8, wherein the veterinary animal is a dog or a cat.
18. The method of any of embodiments 1-8, wherein the livestock is a horse, cow, goat, sheep, pig, or chicken.
19. The method of embodiments 17 or 18, wherein the therapeutically effective amount is 0.1 mg/kg-150 mg/kg.
20. The method of embodiment 19, wherein the therapeutically effective amount is 0.1 mg/kg
50 mg/kg.
21. The method of embodiment 19, wherein the therapeutically effective amount is 2.5 mg/kg
5 mg/kg.
22. The method of any of embodiments 19-21, wherein the therapeutically effective amount is 0.2 mg/kg, 0.4 mg/kg, 1.8 mg/kg, 2.5 mg/kg, 2.6 mg/kg, 5 mg/kg, 10 mg/kg, 15 mg/kg, 20 mg/kg, 30 mg/kg, 40 mg/kg, or 50 mg/kg.
23. Use of mavelertinib as a treatment for giardiasis.
24. A composition including i) mavelertinib in a therapeutically effective amount to treat giardiasis in a subject and ii) a pharmaceutically acceptable carrier.
25. The composition of embodiment 24, formulated for oral administration.
26. The composition of embodiment 25, wherein the oral administration includes a swallowable, chewable, or dissolvable composition.

### (iv) Experimental Example

Repurposing the kinase inhibitor Mavelertinib for giardiasis therapy. Abstract. A phenotypic-based screen of the ReFRAME (Repurposing, Focused Rescue and Accelerated MEdchem) compound-library was performed to identify cell-active inhibitors that could be developed as therapeutics for giardiasis. Primary screen against Giardia lamblia GS-clone H7-strain identified 85 cell-active compounds at a hit rate of 0.72%. A cytotoxicity counter screen against HEK293T was carried out to assess hit compound selectivity for further prioritization. Mavelertinib (PF-06747775), a third-generation epidermal growth factor receptor tyrosine kinase inhibitor (EGFR-TKI), was identified as a potential new therapeutic based on indication, activity, and availability after reconfirmation. Mavelertinib has in vitro efficacy against metronidazole resistant 713-M3 strains. Other EGFR-TKIs screened in follow-up assays exhibited insignificant inhibition of G. lamblia at 5 μM, suggesting that the primary molecular target of Mavelertinib may have a different mechanistic binding mode compared to human EGFR-tyrosine kinase. Mavelertinib, dosed as low as 5 mg/kg or as high as 50 mg/kg, was efficacious in the acute murine Giardia infection model. These results suggest that Mavelertinib merits consideration for repurposing and advancement to giardiasis clinical trials while its analogues are further developed.

Introduction. Despite the enormous public health concern regarding the high prevalence (up to 30%) of giardiasis among malnourished children in economically challenged regions, treatment of severe symptomatic diarrhea and stunting/poor developmental progression associated with asymptomatic giardiasis is often constrained by limited treatment options, marginal efficacy, and/or the development of resistance to available treatments. U.S. Food and Drug Administration approved drugs with efficacy in human giardiasis include metronidazole, tinidazole, nitazoxanide and furazolidone. A substantial number of clinical giardiasis is resistant to these nitro drug treatments. Other potential therapeutics like quinacrine are effective in treating metronidazole-resistant giardiasis but have since been withdrawn from the US market due to poor tolerability. Even with increasing emphasis on new approaches to expand treatment options, development of new drugs against Giardia lamblia is limited. Given the public health need, accelerated development of alternative drugs for clinical use against all Giardia strains, including those with resistance to first-line treatments, is warranted. Recently described advances in G. lamblia phenotypic-based assays and increased access to large collections of well-defined pharmaceutical compound libraries provides new opportunities for identification and repurposing of inhibitors as treatments for giardiasis. The ReFRAME drug-repurposing library, a comprehensive collection of high-value compounds, is an example of advanced platforms for therapeutic discovery to support drug repurposing for neglected diseases. This library is primarily composed of small-molecules that are FDA-approved, undergoing clinical development, or have halted clinical development because clinical endpoints were not achieved.

Screening annotated small-molecule inhibitor libraries including preclinical, clinical or approved drugs can rapidly expedite the discovery of new medicines because hits can be quickly translated from phenotypic assays to proof-of-concept in vivo efficacy models. Here, the ReFRAME library was screened to find potent G. lamblia-inhibiting molecules. The goal was to identify well-defined inhibitors possessing desirable physicochemical properties to accelerate preclinical development against sensitive and resistant strains. The screening effort identified Mavelertinib to be a potent inhibitor of G. lamblia growth and proliferation. Mavelertinib and other related third-generation epidermal growth factor receptor tyrosine kinase inhibitors (EGFR-TKIs) block the proliferation of small cell lung cancer by covalently binding the reactive acrylamide group to a cysteine residue in the ATP-binding domain of mutant EGFR-tyrosine kinase in these cells. Relative to other EGFR-TKIs, Mavelertinib is more potent and has a favorable selectivity index for G. lamblia versus mammalian cell lines.

Mavelertinib effectively clears Giardia infection in a mouse model at clinically relevant doses with no signs of toxicity, suggesting that it could be further developed for more effective treatment of giardiasis.

Materials and Methods. Compound library. The ReFRAME Library's 11,864 inhibitors was used for this study. Metronidazole (Sigma-Aldrich) and Compound-1717 were included as controls. Resupplied compounds were purchased from vendors as high-quality powders. Mavelertinib was purchased from Sigma-Aldrich at ≥98% purity by HPLC. Neratinib, Rociletinib, PD168393, Dacomitinib, AZD8931, AG-18, AG-1478, PD153035-HCl, Erlotinib, Lapatinib Ditosylate, AG-490, Avitinib-Maleate, Gefitinib, and Afatinib were kind gifts from CSN pharm.

Giardia lamblia culture. G. lamblia WB6 strain, G. lamblia CBG99 strain, metronidazole-resistant strain 713-M3 and G. lamblia (Lambl) Alexeieff trophozoites (GS-clone H7; ATCC 50581) used for the primary screening were maintained in modified TYI-S-33 medium (ATCC 2695) and assayed as earlier described (Keister, 1983, Trans R Soc Trop Med Hyg 77: 487-488). Subsequent assays, including the mouse model of infection, used the G. lamblia click beetle green (CBG99) strain which was grown and assayed as previously described (Michaels et al., 2020, J Antimicrob Chemother 75:1218-1227; and Keister, 1983, Trans R Soc Trop Med Hyg 77:487-488).

Experimental compounds and high-throughput screening. The ReFRAME Library-compounds were assayed against axenic G. lamblia GS-clone H7 trophozoites in vitro. The primary screen was conducted at 5 μM. Metronidazole was used at a final concentration of 40 μM as a control for G. lamblia inhibition, and dimethyl sulfoxide (DMSO) at an equivalent percentage (0.25%) was the negative control. All source plates were 384-well acoustic, transfer-compatible plates with compounds pre-diluted in DMSO at either 2 mM or 10 mM. For single-point testing, compounds were transferred into 1536-well tissue culture-treated, white, solid-bottomed, high base microwell plates (Corning 9006BC) with an Echo 555 liquid handle (Labcyte) to a final concentration of 5 μM. For dose response confirmatory testing, compounds were diluted 1:3 in an 8-point titration with a top concentration of either 3 μM or 25 μM. Primary screening was done with a single replicate, whereas dose-response testing was carried out in triplicate.

Compounds were pre-spotted into dry microtiter plates before 5 μL of 250 trophozoites/μL (1250 trophozoites/well) were added with a MultiFlo FX Multi-Mode Dispenser (Biotek). To prevent attachment to the bottle and tubing of the dispenser cassette, the bottle containing the culture was kept on ice throughout the dispensing process. Each assay plate was covered with a universal plastic lid (Greiner) and place into a Bio-Bag Type A (BD #261215). The bags were heat sealed and the anaerobic generator was activated according to manufacturer's instructions. After incubation at 37° C. for 48 hours, 5 μL of BacTiter-Glo (Promega) was added to each well and allowed to develop at room temperature for 5-10 min. Luminescence was read on a ViewLux uHTS Microplate Imager (PerkinElmer) with medium sensitivity luminescence at 30 seconds exposure.

Mammalian cytotoxicity assays. Mammalian cell lines were used for counter screening for general cytotoxicity of hit compounds from the primary screen: human embryonic kidney cells (HEK293T; ATCC CRL-3216) was used in a luminescent ATP assay for detection of cell viability.

Experimental details for the assays were earlier described (Love et al., 2017, PLoS Negl Trop Dis 11:e0005373). Compounds in the follow-up assays were also tested for the inhibition of CRL-8155 (human lymphocytic cells and human hepatocellular carcinoma cells (HepG2; ATCC HB-8065) in a resazurin-based viability assay as indicators of potential host cell toxicity. Assays were performed as previously reported (Vidadala et al., 2014, Eur J Med Chem 74:562-57).

In vitro reconfirmation and expanded tests against EGFR-TKIs using the G. lamblia CBG99 strain were performed as previously described (Michaels et al., 2020, J Antimicrob Chemother 75:1218-1227). Methods for pharmacokinetic studies and analysis of mouse plasma compound concentrations by liquid chromatography with tandem mass spectrometry (LC-MS/MS) have previously been described (Hulverson et al., 2017, J Infect Dis 215:1275-1284; Ojo et al., 2012, J Clin Invest 122:2301-2305; and Schaefer et al., 2016, J Infect Dis 214:1856-1864). The pK studies was performed in groups of 3 mice per compound. Each mouse received a single dose of 50, 20, and 5 mg/kg Mavelertinib by oral gavage. Blood samples collected at intervals between 0.5 and 24 hours were separated and extracted with acetonitrile for measurement of compound concentrations by combined liquid chromatography-electrospray mass spectrometry (LC/ES-MS).

Mavelertinib was tested for efficacy in the luciferase reporter murine infection model using a non-invasive, whole animal IVIS imaging method (Michaels et al., 2020, J Antimicrob Chemother 75:1218-1227). Mice in an experimental group were dosed 50, 20, 5 or 1 mg/kg Mavelertinib QD for 1 to 4 days. All animal experiments were approved by the Institutional Animal Care and Use Committees at the University of Washington.

Data analysis. Relative luminescence units (RLU) values were analyzed in Genedata Screener (v13.0-Standard). RLU was normalized to neutral controls minus inhibitors (DMSO-treated wells minus metronidazole-treated wells). For single-point primary assay plates, an additional run-wise median correction was applied to reduce screen artifacts, whereas no corrections were applied to triplicate dose response plates. For the mammalian cytotoxicity assays, RLU was uploaded into Genedata Screener, and the data was normalized to DMSO-treated wells minus puromycin-treated wells. A four parameter, nonlinear regression curve fit (Smart Fit) was applied to dose response data using Genedata to determine the half maximal effective concentration (Giardia growth inhibition; EC₅₀) or cytotoxic concentration (mammalian cell growth half-maximal cytotoxic concentration; CC₅₀) of each compound. Final filtered hits included those with an EC₅₀≤25 μM and a CC₅₀>10-fold greater than the EC₅₀ value.

Results. High-throughput screen of the ReFRAME Library. The ReFRAME Library was screened against G. lamblia GS-clone H7 strain trophozoite growth by adapting a previously published high-throughput, ATP-based bioluminescence assay in a 1,536-well format (Janes et al., 2018, Proc Natl Acad Sci USA 115:10750-10755; and Chen et al., 2011, Antimicrob Agents Chemother 55:667-675). Compounds were screened at 5 μM with metronidazole (40 μM) as an inhibitor control and DMSO as a neutral control. A cutoff of 50% growth inhibition, as determined from normalized RLU, was applied and resulted in 85 primary hits (0.72% hit rate; Z′-factor 0.73).

These primary hits were immediately assayed for reconfirmation and mammalian cytotoxicity against HEK293T cells in an 8-point dose-response format. Of the 85 primary hits, 57 reconfirmed anti-Giardia activity (EC₅₀<25 μM) and 24 of the reconfirmed hits were selective (CC₅₀>25 μM).

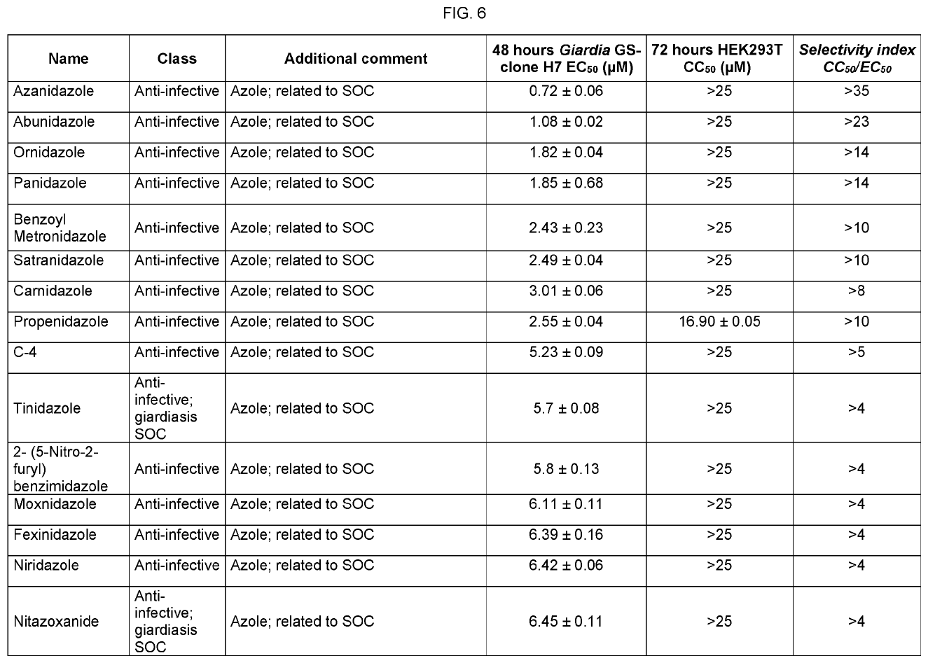

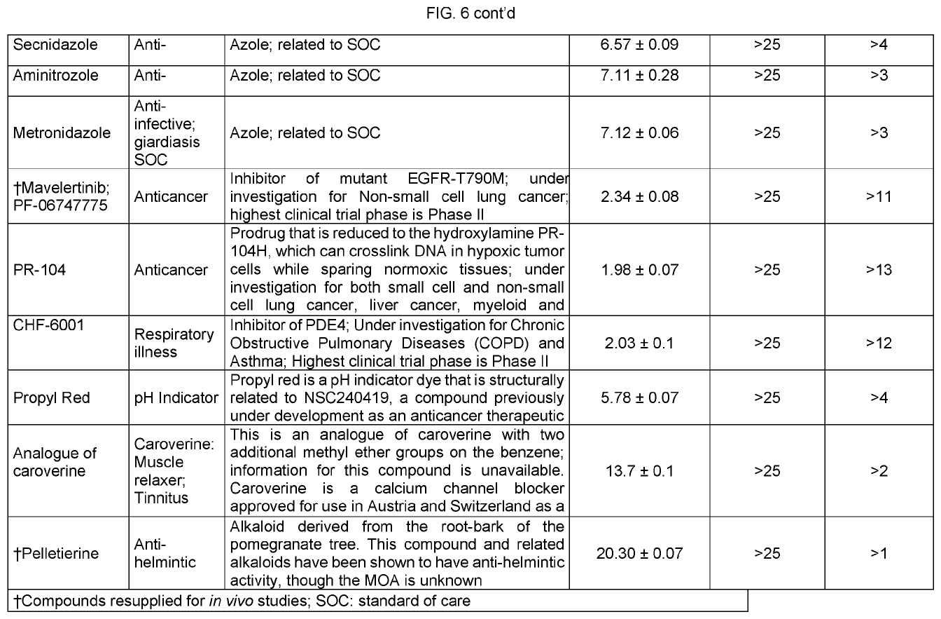

Unsurprisingly, a large proportion of selective hits (18 of 24) included compounds currently in clinical use for giardiasis or those with similar chemical structures (FIG. 6), with only 6 having potentially novel mechanisms of action (MOAs) or new chemotypes for Giardia. The 6 hits are PR-104, CHF-6001, Mavelertinib, propyl red, caroverine and pelletierine. The overall workflow and down selection of hits are summarized in FIG. 1.

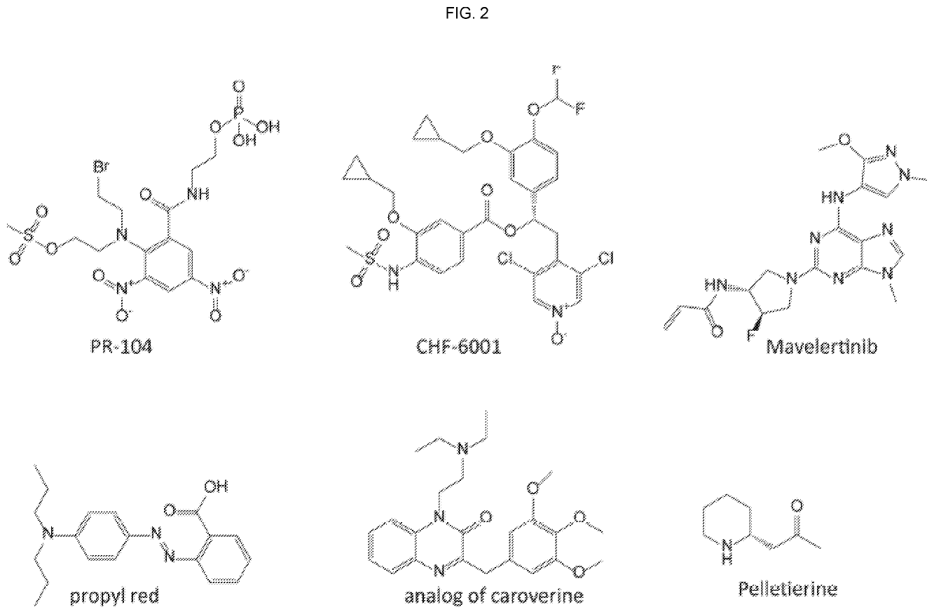

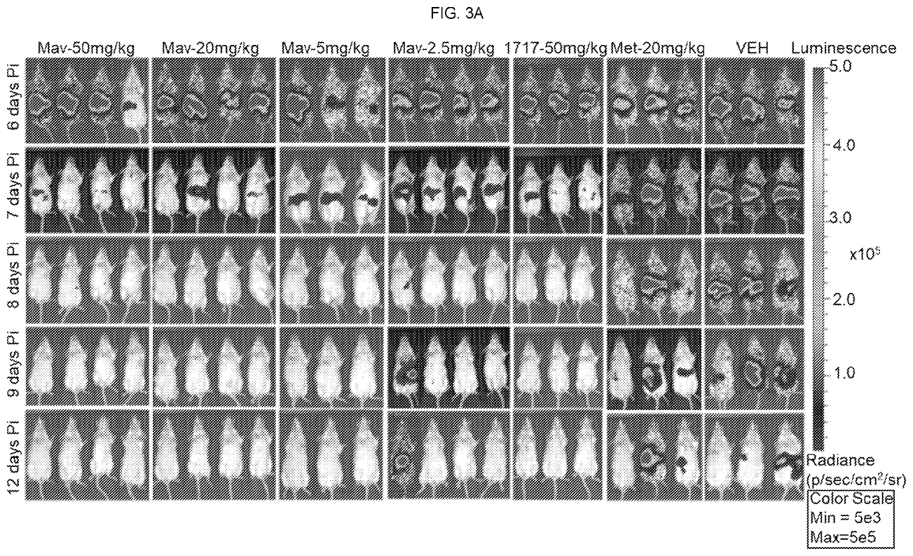

The 6 novel hits (FIG. 2) ranged in potency from 2-20 μM from various indications and uses (FIG. 6). Of these, the 3 most potent compounds (EC₅₀=1.98, 2.34 and 2.03 μM)) included anti-cancer experimental therapeutics PR-104 (Konopleva et al., 2015, Haematologica 100:927-934), Mavelertinib, and the chronic obstructive pulmonary disease/asthma experimental compound, CHF-6001 (Matera et al., 2014, Expert Opin Investig Drugs 23:1267-1275). Two other hits were propyl red and an analogue of the muscle relaxant and tinnitus therapeutic, caroverine (EC₅₀ of 5.78 μM and 13.7 μM against Giardia, respectively). The sixth compound was pelletierine, an alkaloid found in the rootbark of the pomegranate tree (EC₅₀ of 20.3 μM). Pelletierine and related punicine alkaloids have been used for their antihelmintic activity. Compounds for follow-up were selected based on commercial availability and chemical scaffold diversity relative to the standard of care drugs (metronidazole/nitazoxanide). Mavelertinib and pelletierine were resupplied for further studies.

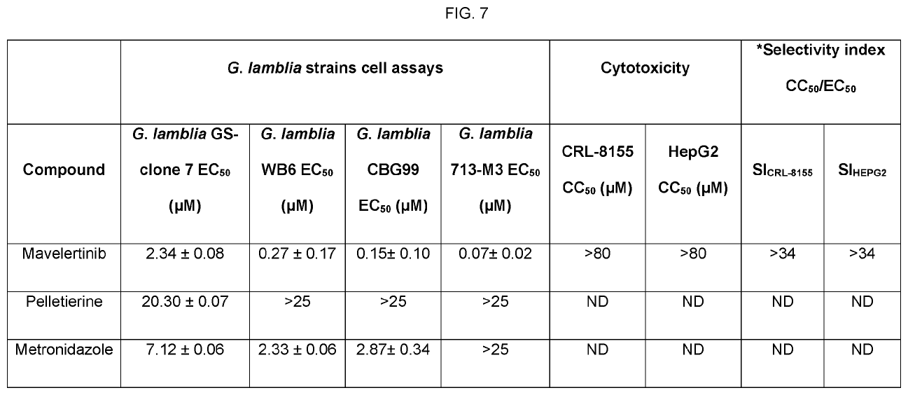

Reconfirmation and test against other EGFR-TKIs. Mavelertinib was reconfirmed using the G. lamblia GS-clone H7 (Assemblage B) and G. lamblia CBG99 (Assemblage A) strains, with resulting EC₅₀ values of 2.34 and 0.15 μM, respectively (FIG. 7). Assemblages are sub-categories of G. lamblia as defined by genomic differences and host variety. The host range from humans, cat, dogs, beavers and guinea-pig for Assemblage A and B to more limited host specific Assemblages. Subsequently, activity against G. lamblia WB6 (Assemblage A) and metronidazole resistant 713-M3 strains was investigated with EC₅₀ values of Mavelertinib being 0.27 and 0.07 μM respectively; while the EC₅₀ value of pelletierine was >25 μM (FIG. 7). The positive control, metronidazole, had a mean EC₅₀ of 2.87 μM against the G. lamblia CBG99 strain. Mavelertinib is, therefore, at least 1.23- to 10-fold more effective against G. lamblia (depending on the strain) relative to metronidazole. Cytotoxicity assays of Mavelertinib against CRL-8155 and HepG2 cells showed CC₅₀ values of >80 μM (FIG. 7). Based on these results, pelletierine was deprioritized and focus was shifted to Mavelertinib.

To investigate anti-Giardia activity with compounds similar to Mavelertinib, 14 additional human EGFR-TKIs were sourced for screening against G. lamblia CBG99 at 5 μM. These included Neratinib, Rociletinib, AZD8931, Afatinib, Avitinib-maleate, AG-490, Lapatinib Ditosylate, PD168393, PD153035-HCl, AG-18, Dacomitinib, AG-1478, Gefitinib, and Erlotinib, representing different developmental generations with and without the potential for covalent binding.

Unfortunately, none of the additional EGFR-TKIs inhibited G. lamblia growth above 50% at 5 μM, suggesting that the mechanism of inhibition of the molecular target in Giardia may be distinct from the mechanism used to inhibit human EGFR-tyrosine kinase, and a targeted structure activity relationship study is warranted in the future.

Pharmacokinetics and efficacy of Mavelertinib in a mouse model of infection Mavelertinib was investigated for in vivo efficacy in an acute mouse infection model using the G. lamblia CBG99 strain (Michaels et al., 2020, J Antimicrob Chemother 75:1218-1227). This strain is useful for non-invasive, quantitative measurement of antimicrobial treatment effects in vivo. The method relies on a stable constitutive reporter, CBG99, that correlates with total parasite load. Infection in female BALB/c or B6 IFN-γ KO mice is clearly detectable with the IVIS® (Xenogen Corporation, Alameda, CA) In Vivo Imaging System (PerkinElmer) by 5 days post-infection and persists for more than 2 weeks. In the model, vehicle treated mice have persistent infection, whereas successfully treated mice appear to clear the infection. Reliability of the model to measure in vivo efficacy was previously validated using methionyl-tRNA synthetase inhibitor Compound-1717 and was therefore used as a control along with metronidazole in the experiments reported here. Of note, metronidazole was administered at 20 mg/kg orally once per day (QD) for the treatment period, an insufficient regimen that will not clear the infection within the dosing timeline.

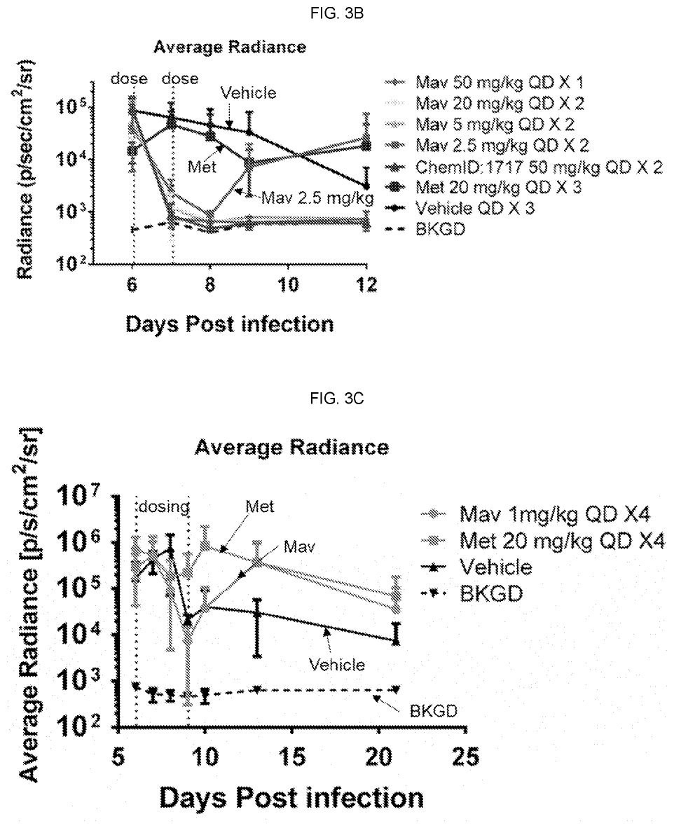

Mavelertinib dosed at 50 mg/kg QD for 3 days appeared to clear the infection (data not shown). In subsequent experiments with one- and two-day dosing of 50 mg/kg QD Mavelertinib, all treated mice were cleared of infection and remained clear a week after dosing. Mavelertinib was further profiled in a dose-response experiment with the following regimens: 50 mg/kg QD×1 day, 20 mg/kg QD×2 days, 5 mg/kg QD×2 days, and 2.5 mg/kg QD×2 days. Except for the 2.5 mg/kg group, where one of four mice remained infected, all regimens cleared the infection below the background signal (FIG. 3). A dosage regimen of 1 mg/kg QD×4 days was subsequently tested for efficacy.

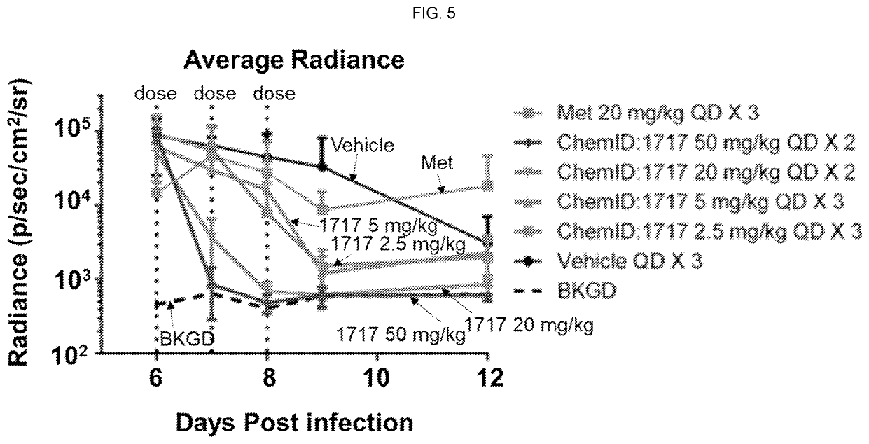

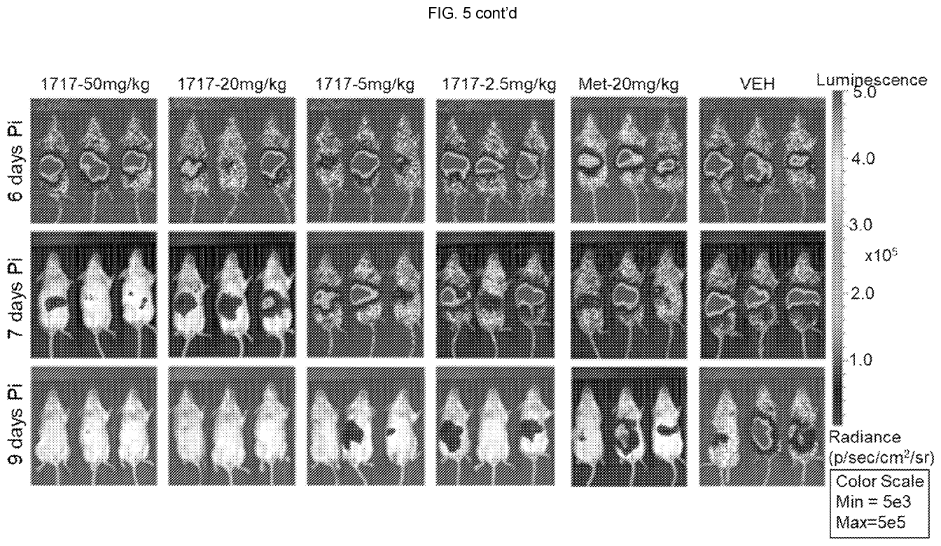

None of the 1 mg/kg QD×4 days mice were relieved of their infection (FIG. 3C). The lack of luminescence signals above background levels (FIGS. 3A and 3B) one week after the last of the therapeutic doses confirmed that parasites did not rebound after treatment with Mavelertinib. Mavelertinib was therefore shown to be efficacious in a dose-dependent manner in an acute murine infection model, with 50 mg/kg QD dose administered once being as effective as some of the lower doses administered over longer periods. For Compound-1717 treated control groups, 50 and 20 mg/kg QD×2 days cleared the infection, whereas the 5 mg/kg and 2.5 mg/kg QD×3 days did not. In both latter cases, only one of three mice appeared to be cleared of infection (FIGS. 3A and 5). The 20 mg/kg QD×3 days metronidazole and vehicle groups all remained infected after treatment.

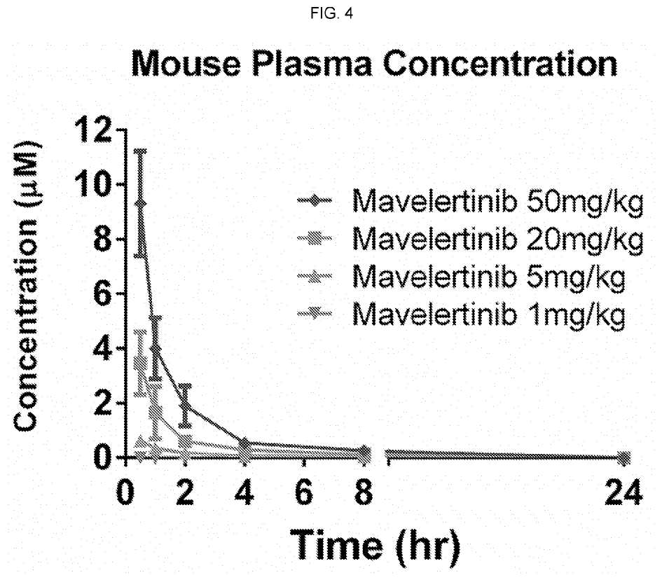

In all Mavelertinib treatment groups, plasma trough levels taken on days 6 and 7 post-infection (24 hours and 48 hours after the first dose) were below the limit of quantitation. Single dose mouse pharmacokinetic studies suggest that Mavelertinib is cleared from plasma by 24 hours (FIG. 4), which reflects what was seen in the Giardia efficacy experiments in which there was no accumulation of Mavelertinib in the plasma.

The 50 mg/kg dose had an area-under-the-curve (AUC) of 812 min*μmol/L and an average maximum plasma concentration (Cₘₐₓ) of 9.3 μM with the average volume of blood plasma cleared of drug per unit time (Clearance) being 0.0016 L/min. The 20 mg/kg and 5 mg/kg Cₘₐₓ were 3.5 μM and 0.63 μM, with AUCs of 300 and 89 min*μmol/L, respectively, and clearances of 0.004 L/min and 0.015 L/min, respectively.

The time (Tₘₐₓ) at which Cₘₐₓ is observed was 0.5 hours in all cases while average half-life (t1/2) was 125.6, 174.8 and 239.2 minutes for the 50 mg/kg, 20 mg/kg and 5 mg/kg dosages respectively. Mavelertinib, when dosed 1 mg/kg QD was barely detectable in the plasma of one mouse at 0.5 hours post dose and below the limit of detection at all subsequent time points. Clinical evaluation of overall health of all mice used for in vivo PK and efficacy studies suggests no observable mavelertinib induced toxicology potentials.

The observed systemic exposure may or may not be directly linked to in vivo efficacy since it is uncertain whether Mavelertinib was delivered to Giardia via the bloodstream or directly from the intestinal lumen. Hence, pharmacodynamic properties associated with in vivo efficacy could be pliable if toxicity to the host is minimal and exposure at the site of infection is sufficient for treating the disease. Even so, it appears that short courses of high doses may clear infection faster than low doses sustained over time for Mavelertinib.

Discussion. In addition to the discussed limitations, including reduced efficacy and adverse side-effects, many available giardiasis drugs also have broad spectrum antimicrobial activity that may lead to the development of gut dysbiosis. Gut dysbiosis can complicate giardiasis treatment in malnourished children, a major population group for Giardia therapeutics. Given its high prevalence in resource limited regions, giardiasis is a neglected infectious disease that could benefit from additional treatment options based on drug repurposing platforms that will address these concerns.

Until recently, minimal effort has been dedicated to developing new anti-infectives against G. lamblia, despite the great clinical need. Renewed interest in giardiasis therapeutic discovery has spiked due to advances in G. lamblia reporter systems that are ideal for whole cell high-throughput screens and in vivo screening of pharmaceutical libraries. The ReFRAME library could accelerate the process of finding new treatments against Giardiasis and other infectious parasitic diseases afflicting millions but have limited drug discovery and development resources. With 37 compounds from pharmaceutical companies approved for various human clinical uses and >250 currently in clinical trials, finding kinase inhibitors that could be readily repurposed for treating giardiasis and other indications among this pool is highly desirable. Giardia protein kinases are not specifically targeted by any available giardiasis drugs, making inhibitors of these enzymes attractive agents for therapeutic development against sensitive and resistant strains.

Identifying inhibitors and optimizing drug candidates for selective inhibition of parasite targets is challenging due to high functional and structural similarities between many mammalian and parasite essential enzymes. This concern could significantly dampen enthusiasm for anti-giardiasis drug development programs based on drug repurposing platforms. However, evolutionary drift that often results in subtle but essential differences in sequences or conformation may be exploited to achieve selectivity and clinical relevance. Mavelertinib belongs to a new generation of EGFR-TKIs that selectively target mutation of EGFR gatekeeper residue 790 (T790M) and other oncogenic mutations, thereby reducing safety risks. This may be the reason for its high selectivity index for Giardia versus mammalian cells and lack of adverse effects in mouse treatment experiments.

Research studies to decipher conformity or diversity in the degree of susceptibility to specific drugs among G. lamblia strains and assemblages have been vitiated by conflicting conclusions. Nevertheless, the same drugs are used to effectively treat all Giardia-associated diseases, which suggests a high level of homogeneity. Mavelertinib inhibitory effects on G. lamblia assemblages A and B as well as metronidazole resistant 713-M3 strain demonstrated here could therefore potentially be extended to other strains. Intriguingly, the primary molecular target in Giardia has yet to be defined. While knowledge of a well-defined primary molecular target is highly desirable, it may not be essential for product development in this case since Mavelertinib is a repurposed agent with well-defined safety profiles. Nonetheless, biochemical, genomic and chemical-genetic studies including selections of resistance, overexpression, RNA interference and morpholino knockdown of protein expression to determine the target are ongoing.

Mavelertinib shows properties consistent with preclinical candidates for treatment of giardiasis and has already entered human Phase II clinical trials for lung cancer. Its chemical synthesis, safety profiles, and a partial analysis of pharmacokinetic properties have been described. As part of Mavelertinib's Phase I clinical trials (NCT02349633), dose escalation studies in humans showed that administration of >150 mg with daily oral doses over 7 days in adults caused diarrhea and skin toxicities as the most common adverse events. Given that the average adult weight worldwide ranges from 57.7 kg in Asia to 80.7 kg in North America, a 150 mg dose equates to 2.6-1.9 mg/kg. Allometric scaling of the efficacious doses of 5 and 2.5 mg/kg in mice to the human equivalent dose (0.081×mouse dose in mg/kg) yields dose predictions of between 0.4 mg/kg and 0.2 mg/kg needed for average weight adult humans. This leaves a possible safety window of 4.6 to 13 times the efficacious dose, depending on the dose needed and the weight of the adult patient. Since the equivalent human efficacious dose (based on the murine efficacy data) is below the clinical trial threshold and the drug may not necessitate more than 7 daily doses, all previously determined GLP toxicological data associated with the Phase I clinical trials remain relevant and may not need to be repeated. The direct implication for Mavelertinib's potential use as an anti-giardiasis is that it can proceed to clinical trial Phase II, thereby fulfilling the goal of identifying and accelerating preclinical development of drugs against pathogens for which treatment options are limited or compromised by development of antimicrobial resistance.

Reduced cost of development should have a direct impact on the cost of goods for the target population. This is certainly true for G. lamblia.

In the final analysis, a significant percentage of the population of interest is malnourished children, which underscores the need to investigate new leads for an improved safety index. Pfizer previously published detailed medicinal chemistry optimizations of the Mavelertinib scaffold, along with several EGFR crystal structures in complex with Mavelertinib and its analogues.

### (v) Closing Paragraphs

Unless otherwise indicated, the practice of the present disclosure can employ techniques of chemistry, organic chemistry, biochemistry, analytical chemistry, and physical chemistry. These methods are described in the following publications. See, e.g., Harcourt, et al., Holt McDougal Modern Chemistry: Student Edition (2018); J. Karty, Organic Chemistry Principles and Mechanisms (2014); Nelson, et al., Lehninger Principles of Biochemistry 5th edition (2008); Skoog, et al., Fundamentals of Analytical Chemistry (8th Edition); Atkins, et al., Atkins' Physical Chemistry (11th Edition).

Each embodiment disclosed herein can comprise, consist essentially of, or consist of its particular stated element, step, ingredient, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. A material effect would cause a statistically significant reduction in the ability of malvelertinib disclosed herein to inhibit giardiasis and/or reduce or prevent giardiasis.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing the disclosure (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. There is an expectation that skilled artisans employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents, printed publications, journal articles, and other written text throughout this specification (referenced materials herein). Each of the referenced materials are individually incorporated herein by reference in their entirety for their referenced teaching.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice.

Definitions and explanations used in the present disclosure are meant and intended to be controlling in any future construction unless clearly and unambiguously modified in the examples or when the application of the meaning renders any construction meaningless or essentially meaningless. In cases where the construction of the term would render it meaningless or essentially meaningless, the definition should be taken from Webster's Dictionary, 3rd Edition, or a dictionary known to those of ordinary skill in the art, such as the Oxford Dictionary of Biochemistry and Molecular Biology (Eds. Attwood T et al., Oxford University Press, Oxford, 2006).

1.-26. (canceled)

27. A method of treating giardiasis in a subject in need thereof comprising administering a therapeutically effective amount of a composition comprising mavelertinib to the subject, thereby treating giardiasis in the subject in need thereof.

28. The method of claim 27, wherein the administering is a single administration.

29. The method of claim 27, wherein the administering is two, three, four, five, six, or seven administrations.

30. The method of claim 29, wherein the two, three, four, five, six, or seven administrations are each on a consecutive day.

31. The method of claim 27, wherein the administering comprises oral administration.

32. The method of claim 27, wherein the subject is a human, a veterinary animal, or a livestock.

33. The method of claim 27, wherein the subject is a human.

34. The method of claim 33, wherein the therapeutically effective amount is 0.1 mg/kg-2.6 mg/kg.

35. The method of claim 33, wherein the therapeutically effective amount is 0.2 mg/kg, 0.3 mg/kg, 0.4 mg/kg, 0.5 mg/kg, 0.6 mg/kg, 0.8 mg/kg, 1 mg/kg, 1.2 mg/kg, 1.4 mg/kg, 1.6 mg/kg, 1.9 mg/kg, or 2.6 mg/kg.

36. The method of claim 32, wherein the veterinary animal is a dog or a cat.

37. The method of claim 36, wherein the therapeutically effective amount is 0.1 mg/kg-150 mg/kg.

38. The method of claim 36, wherein the therapeutically effective amount is 0.1 mg/kg-50 mg/kg.

39. The method of claim 36, wherein the therapeutically effective amount is 0.2 mg/kg, 0.4 mg/kg, 1.8 mg/kg, 2.5 mg/kg, 2.6 mg/kg, 5 mg/kg, 10 mg/kg, 15 mg/kg, 20 mg/kg, 30 mg/kg, 40 mg/kg, or 50 mg/kg.

40. The method of claim 32, wherein the livestock is a horse, cow, goat, sheep, pig, or chicken.

41. The method of claim 40, wherein the therapeutically effective amount is 0.1 mg/kg-150 mg/kg.

42. The method of claim 40, wherein the therapeutically effective amount is 0.1 mg/kg-50 mg/kg.

43. The method of claim 40, wherein the therapeutically effective amount is 0.2 mg/kg, 0.4 mg/kg, 1.8 mg/kg, 2.5 mg/kg, 2.6 mg/kg, 5 mg/kg, 10 mg/kg, 15 mg/kg, 20 mg/kg, 30 mg/kg, 40 mg/kg, or 50 mg/kg.

44. A method of treating giardiasis in a subject in need thereof comprising administering 0.1 mg/kg-50 mg/kg mavelertinib to the subject, thereby treating giardiasis in the subject in need thereof.

45. A composition comprising i) mavelertinib in a therapeutically effective amount to treat giardiasis in a subject and ii) a pharmaceutically acceptable carrier.

46. The composition of claim 45, formulated for oral administration.
